SEQUENCE LISTING (2 sequences)

SEQ ID NO: 1 - DNA, 19 bp, Artificial sequence
  synthetic
gatggtgcagccgctatta

SEQ ID NO: 2 - DNA, 24 bp, Artificial sequence
  synthetic
ggtgggtgtgggtataatactaag

(19) United States
(12) Patent Application Publication
(10) Pub. No.: US 2023/0203592 A1
(43) Pub. Date: Jun. 29, 2023

(54) COMPOSITIONS AND METHODS FOR CHARACTERIZING BOWEL CANCER

(71) Applicant: Akershus Universitetssykehus HF, Lørenskog (NO)

(72) Inventors: Anne Hansen REE, Haslum (NO); Anna Paula BOUSQUET, Donsö (SE); Tonje BJØRNETRØ, Oslo (NO); Sebastian MELTZER, Oslo (NO); Kathrine Røe REDALEN, Trondheim (NO)

(21) Appl. No.: 17/923,331

(22) PCT Filed: May 5, 2021
(86) PCT No.: PCT/IB2021/000298

§ 371 Date: Nov. 4, 2022

Related U.S. Application Data
(60) Provisional application No. 63/020,333, filed on May 5, 2020.

(51) Int. Cl. C12Q 1/6886

(52) U.S. Cl. CPC C12Q 1/6886; C12Q 2600/156; C12Q 1/6844

(57) ABSTRACT

The present invention relates to compositions and methods for characterizing cancer. In particular, the present invention relates to compositions and methods for identifying bowel cancers at increased risk of metastasis.

### CROSS-REFERENCE TO RELATED APPLICATION

The present application claims priority to U.S. Provisional Patent Application No. 63/020,333, filed May 5, 2020, which is hereby incorporated by reference in its entirety.

### FIELD OF THE INVENTION

The present invention relates to compositions and methods for characterizing cancer. In particular, the present invention relates to compositions and methods for identifying bowel cancers at increased risk of metastasis.

### BACKGROUND OF THE INVENTION

Colorectal cancer (CRC; cancer in the large bowel) is common in both sexes, with an increasing incidence owing to the aging population. CRC is a heterogeneous disease of high biological complexity,¹ which calls for increasingly individualized treatment based on biological characteristics.² About half of CRC patients develop metastasis (spread of the disease to other body organs). Metastatic CRC, particularly in abdominal cavity organs, remains the main cause of severe morbidity and dismal survival.³ New diagnostic developments provide a direction to the next milestone in CRC management, which is the control of early metastatic progression, typically as spread to the liver.

CRC adenocarcinoma that is localized within the abdominal or pelvic cavity indicates that patients may be cured by surgery. In the current routine practice, assessment of metastatic risk is based on imaging modalities (e.g., CT and/or MR scanning).

If metastatic risk is considered low, the CRC patient proceeds directly to the surgical procedure. Based on histologic findings in the surgical specimen, the patients may receive post-operative (adjuvant) chemotherapy or radiotherapy. However, a considerable percentage of patients are treated unnecessarily as it is not known who remains with subclinical metastasis or not after the surgery.

If the primary rectal cancer is considered to be at high risk of metastasizing, the patient receives pre-operative (neoadjuvant) (chemo)radiotherapy, which commonly comes with considerable side-effects during the treatment and long-term sequelae. Despite the neoadjuvant therapy, which has led to significantly improved local recurrence rates,⁸ still as many as 30-40% of patients experience distant metastasis.⁹⁻¹¹ The addition of adjuvant chemotherapy in this setting has not been convincing.^{11,12} Recent efforts have been made to improve outcome by the addition of induction or consolidation chemotherapy within the neoadjuvant treatment course, the concept of total neoadjuvant therapy.¹³ However, tools for the optimal selection of patients to the new treatment strategies are needed.

### SUMMARY OF THE INVENTION

The present invention relates to compositions and methods for characterizing cancer. In particular, the present invention relates to compositions and methods for identifying bowel cancers at increased risk of metastasis.

The compositions and methods described herein improve patient care by identifying individuals in need of additional therapies and providing such therapies only to those in need.

For example, in some embodiments, provided herein is a method of identifying the presence of a mtDNA variant in a sample from a subject diagnosed with colorectal cancer (CRC; e.g., metastatic CRC), comprising: a) contacting the sample with one or more reagents specific for detecting the presence of one or more variations in the MT-RNR2 gene; and b) determining the presence of the variations in the sample. The present disclosure is not limited to particular variants of MT-RNR2. Examples include, but are not limited to, 3105AC>A and/or 3106CN>C.

Further embodiments provide a method of treating CRC, comprising: a) determining the presence of one or more variations in the MT-RNR2 gene in a sample from a subject diagnosed with CRC, wherein the variations are 3105AC>A and/or 3106CN>C; and b) administering neo/adjuvant therapy (e.g., one or more of chemotherapy, radiotherapy, targeted therapy, or immunotherapy) to subjects with the absence of a 3105AC>A variation and/or the presence of a 3106CN>C variation.

Yet other embodiments provide a method of determining an increased risk of a CRC patient having metastasis, comprising: a) determining the presence of one or more variations in the MT-RNR2 gene in a sample from a subject diagnosed with CRC, wherein the variations are 3105AC>A and/or 3106CN>C; and b) identifying the subject as having an increased risk of metastasis when the sample has the absence of the 3105AC>A variation and/or the presence of the 3106CN>C variation. In some embodiments, the method further comprises the step of administering adjuvant chemotherapy (e.g., one or more of chemotherapy, radiotherapy, targeted therapy, or immunotherapy) to subjects with the absence of a 3105AC>A variation and/or the presence of a 3106CN>C variation.

Any number of suitable methods may be utilized to identify variants of MT-RNR2. Examples include, but are not limited to, amplifying and/or sequencing the MT-RNR2 gene. In some embodiments, the amplifying is digital PCR. Exemplary reagents for use in the detection methods include, but are not limited to, one or more sequencing primers, one or more amplification primers or one or more nucleic acid probes. In some embodiments, the reagents further comprise one or more restriction enzymes. In some preferred embodiments, the digital PCR methods of the present invention utilize a restriction enzyme digestion of the target and/or template DNA. Thus, in some embodiments, the method of the present invention utilize enzymatic restriction of template DNA isolated from a suitable source such as blood or EVs. In particular, in some embodiments, the template DNA sample is treated with either EarI or AgsI. The EarI restriction enzyme recognizes the wild-type (non-mutated) 3105 site of MT-RNR2, while the AgsI restriction enzyme recognizes a mutation in the 3106 site of MT-RNR2. Hence, a high percentage of non-digested product is expected by a PCR containing EarI if the mutation (point deletion) 3105AC>C is present, while for AgsI, the mutation (point deletion) 3106CN>C will cause a lower percentage of the non-digested product. The relative percentages of the wild-type or mutated positions may preferably be determined by digital PCR. A subject may be statified as being at an increased risk of metastasis when the sample has the absence of the 3105AC>A variation and/or the presence of the 3106CN>C variation. In some embodiments, the sample is, for example, whole blood (WB) or an isolated fraction of extracellular vesicles (EV).

Additional embodiments are described herein.

### DESCRIPTION OF THE FIGURES

FIG. 1. Left panel: The number of cases with either the wild-type homoplasmic variant (heteroplasmy 0) or the AC>A deletion variant (heteroplasmy of 0.964-0.995) of the WB-mtDNA 3105 site. Open circles: no progression. Closed circles: metastatic progression. Right panel: Progression-free survival (survival without metastatic progression) in the two patient groups; p=0.016 by log-rank test.

FIG. 2. Left panel: The number of cases with either the wild-type homoplasmic variant (heteroplasmy 0) or the CN>C deletion variant (heteroplasmy of 0.893-0.998) of the WB-mtDNA 3106 site. Open circles: no progression. Closed circles: metastatic progression. Right panel: Progression-free survival in the two patient groups; p=0.016 by log-rank test.

### DEFINITIONS

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “subject” refers to any organisms that are screened using the diagnostic methods described herein. Such organisms preferably include, but are not limited to, mammals (e.g., humans).

The term “diagnosed,” as used herein, refers to the recognition of a disease by its signs and symptoms, or genetic analysis, pathological analysis, histological analysis, and the like.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, those described herein.

As used herein, the term “characterizing cancer in a subject” refers to the identification of one or more properties of a cancer sample (e.g., including but not limited to, the presence of cancerous tissue, the presence or absence of variants or mutations in mtDNA, the presence of pre-cancerous tissue that is likely to become cancerous, and the presence of cancerous tissue that is likely to metastasize).

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues (e.g., biopsy samples), cells, vesicles, and gases. Biological samples include blood products, such as plasma, serum and the like. Such examples are not however to be construed as limiting the sample types applicable to the present invention.

### DETAILED DESCRIPTION OF THE INVENTION

Biological processes participating in the interrelation between cancer and the immune system are important for understanding and defining prognosis.¹ Tumor-defeating immunity entails the activation of cytolytic lymphocytes (killer CD8⁺ T-cells), but protective mechanisms against auto-immunity (immune attack on the organism's healthy tissues) impede the immune surveillance and create immune tolerance to the cancer. The role of the immune cell energy metabolism in surveillance versus tolerance currently draws increasing attention; particularly, activated T-cells have an enormous demand for energy when they exponentially proliferate to mount efficient immunity.⁴

A cell's metabolism is a result of the mitochondrial function. Mitochondria are intracellular organelles containing their own DNA (mtDNA). The mtDNA genome is a circular molecule, only 16,569 bases long, encoding subunits of enzyme complexes that drive the metabolism. Each mammalian cell may harbor 100 or more mitochondria, each with numerous mtDNA copies. Because the mutation frequency of replicating mtDNA is high, mutant mtDNA copies are often mixed with normal (wild-type) copies within the cell. At their discovery in the 1980s, mtDNA polymorphisms (sequence variants/mutations) were thought not to have any phenotypic (functional) effects, but it soon became evident that they can alter mitochondrial function, particularly in cells that are highly dependent on the metabolism. Nevertheless, if a mutation is pathogenic, the cell can commonly tolerate a high percentage level of this mtDNA variant before the biochemical threshold is exceeded with resulting metabolic defects.⁵

Recent reports from experimental laboratory models have demonstrated that the entire mtDNA genome can be packed inside EVs,^{6,7} which are small particles that are naturally released from most cell types into body fluids.

Knowledge about the metastatic propensity of CRC has clear therapeutic consequences. Over the past decade, improved multimodal therapy approaches have enabled an increasing number of patients with liver-confined metastatic disease to undergo treatment with potentially curative intent, although metastatic recurrence occurs in a high percentage of cases. New biological markers contribute to better selection of patients at an earlier time within the course of the disease, while it is still subclinical, for more intensified therapies with the intent to cure more CRC patients at high metastatic risk. Likewise, patients with CRC that is unable to metastasize can be spared oncologic therapy before or after surgery, which commonly comes with adverse side-effects, and the intense follow-up program with repeat CT scans over years after the surgery, which is also advantageous with regard to an optimized resource use within the specialist healthcare. Patients with high-risk CRC are a group that continues to expand owing to the aging population.

Accordingly, provided herein are compositions and methods for identifying CRC patients with increased risk of metastasis and disease progression. For example, in some embodiments, provided herein is a method of determining an increased risk of a CRC patient having metastasis, comprising: a) determining the presence of one or more variations in the mtRNA (e.g., MT-RNR2 gene) in a sample from a subject diagnosed with CRC, wherein the variations are, for example, 3105AC>A and/or 3106CN>C; and b) identifying the subject as having an increased risk of metastasis (e.g., when the sample has the absence of the 3105AC>A variation and/or the presence of the 3106CN>C variation).

In some embodiments, the results are used to determine a treatment course of action. For example, in some embodiments, neoadjuvant or adjuvant chemotherapy (e.g., one or more of chemotherapy, radiotherapy, targeted therapy, or immunotherapy) is administered to subjects at increased risk of disease progression (e.g., subjects with an absence of a 3105AC>A variation and/or the presence of a 3106CN>C variation).

Any number of suitable methods may be utilized to identify variants of MT-RNR2. Examples include, but are not limited to, amplifying and/or sequencing the MT-RNR2 gene. In some embodiments, the amplifying is digital PCR. Exemplary reagents for use in the detection methods include, but are not limited to, one or more sequencing primers, one or more amplification primers or one or more nucleic acid probes. In some embodiments, the reagents further comprise one or more restriction enzymes.

In some embodiments, the sample is, for example, whole blood (WB) or an isolated fraction of extracellular vesicles (EV).

Exemplary detection methods are described herein.

Nucleic acids may be amplified prior to or simultaneous with detection. Illustrative non-limiting examples of nucleic acid amplification techniques include, but are not limited to, polymerase chain reaction (PCR), digital PCR, reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

Digital PCR, or dPCR, dPCR involves partitioning the PCR solution into tens of thousands of nano-liter sized droplets, where a separate PCR reaction takes place in each one (See e.g., Duewer, David L.; et al. (2018). Analytical and Bioanalytical Chemistry. 410 (12): 2879-2887; Baker, Monya (2012). Nature Methods. 9 (6): 541-544; each of which is herein incorporated by reference in its entirety). Several different methods can be used to partition samples, including microwell plates, capillaries, oil emulsion, and arrays of miniaturized chambers with nucleic acid binding surfaces (Quan, Phenix-Lan et al., (2018). Sensors. 18 (4): 1271). The PCR solution is divided into smaller reactions and are then made to run PCR individually. After multiple PCR amplification cycles, the samples are checked for fluorescence with a binary readout of “0” or “1”. The fraction of fluorescing droplets is recorded. The partitioning of the sample allows one to estimate the number of different molecules by assuming that the molecule population follows the Poisson distribution, thus accounting for the possibility of multiple target molecules inhabiting a single droplet. Using Poisson's law of small numbers, the distribution of target molecule within the sample can be accurately approximated allowing for a quantification of the target strand in the PCR product. This model simply predicts that as the number of samples containing at least one target molecule increases, the probability of the samples containing more than one target molecule increases. In some embodiments, commercially available dPCR partitioning, amplification, and analysis systems are utilized (e.g., available from Bio-Rad, Hercules, Calif.).

In some preferred embodiments, the digital PCR methods of the present invention utilize a restriction enzyme digestion of the target and/or template DNA. Thus, in some embodiments, the method of the present invention utilize enzymatic restriction of DNA isolated from a suitable source such as blood or EVs. In particular, in some embodiments, the DNA sample is treated with either EarI or AgsI. The EarI restriction enzyme recognizes the wild-type (non-mutated) 3105 site of MT-RNR2, while the AgsI restriction enzyme recognizes a mutation in the 3106 site of MT-RNR2. Hence, a high percentage of non-digested product is expected by a PCR containing EarI if the mutation (point deletion) 3105AC>C is present, while for AgsI, the mutation (point deletion) 3106CN>C will cause a lower percentage of the non-digested product. The relative percentages of the mutations can be determined by digital PCR.

A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

In some particularly preferred embodiments, DNA sequencing methodologies associated with the present technology comprise Second Generation (a.k.a. Next Generation or Next-Gen or NGS), Third Generation (a.k.a. Next-Next-Gen), or Fourth Generation (a.k.a. N3-Gen) sequencing technologies including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the present technology provides parallel sequencing of partitioned amplicons (PCT Publication No: WO2006084132 to Kevin McKernan et al., herein incorporated by reference in its entirety). In some embodiments, DNA sequencing is achieved by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; U.S. Pat. Nos. 6,432,360, 6,485,944, 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. Nos. 6,787,308; 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbial., 7:287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa and Nextera platforms commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbial., 7: 287-296; U.S. Pat. Nos. 6,210,891; 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbial., 7:287-296; U.S. Pat. Nos. 6,833,246; 7,115,400; 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

In some embodiments, nucleic acid sequencing methods comprise methods and reagents for tagmenting a sample of nucleic acid (e.g., mitochondrial genomic DNA). Suitable tagmentation reagents include, for example, those provided by Illumina in the NEXTERA DNA or NEXTERA DNA Flex library preparation kit. The transposomes are utilized to fragment the nucleic acid samples at approximately 250 to 1,500 bp in length, more preferably from 200 to 400 bp intervals and most preferably at about 300 bp intervals. As part of the tagmentation reaction, transposon adapter sequences are added to the 5′ ends of the sequence fragments. In the normal Nextera protocol, indexed sequencing primers that anneal to the adapter sequences are used in a limited cycle PCR to amplify the fragments to make a library for sequencing. Suitable NEXTERA reagents and methods are described in the following US Patents, which are all incorporated herein by reference in their entirety: U.S. Pat. Nos. 7,303,901; 9,040,256; 9,080,211; 9,085,801; 9,115,396; 9,683,230; 9,828,627; 10,041,066; 10,184,122; and 10,525,437. In some embodiments, the NEXTERA reagents are used in conjunction with the MISEQ sequencing reagents as described in the following US patents, which all incorporated by reference herein their entirety: U.S. Pat. Nos. 7,057,026; 7,329,860; 7,414,116; 7,427,673; 7,541,444; 7,589,315; 7,592,435; 7,795,424; 7,816,503; 7,960,685; 8,039,817; 8,071,962; 8,084,590; 8,158,926; 8,212,015; 8,241,573; 8,244,479; 8,315,817; 8,394,586; 8,412,467; 8,460,910; 8,563,477; 8,852,910; 8,914,241; 8,951,781; 8,965,076; 9,068,220; 9,121,063; 9,365,898; 9,512,422; 9,765,309; 9,970,055; 10,017,750; 10,220,386; 10,227,636; 10,480,025; 10,487,102; and 10,519,496.

Sequencing nucleic acid molecules using SOLiD technology (Voelkerding et al., Clinical Chem., 55:641-658, 2009; MacLean et al., Nature Rev. Microbial., 7:287-296; U.S. Pat. Nos. 5,912,148; 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (see, e.g., Astier et al., J. Am. Chem. Soc. 2006 Feb. 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, HeliScope by Helicos BioSciences is employed (Voelkerding et al., Clinical Chem., 55: 641-658, 2009; MacLean et al., Nature Rev. Microbiol., 7:287-296; U.S. Pat. Nos. 7,169,560; 7,282,337; 7,482,120; 7,501,245; 6,818,395; 6,911,345; 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is .about.99.6% for 50 base reads, with about 100 Mb generated per run. The read-length is 100 base pairs. The accuracy for homopolymer repeats of 5 repeats in length is about.98%.

Another exemplary nucleic acid sequencing approach that may be adapted for use with the present invention was developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “HIGH THROUGHPUT NUCLEIC ACID SEQUENCING BY EXPANSION,” filed Jun. 19, 2008, which is incorporated herein in its entirety.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectable fluorescence resonance energy transfer (FRET) upon nucleotide addition.

Another real-time single molecule sequencing system developed by Pacific Biosciences (Voelkerding et al., Clinical Chem., 55: 641-658, 2009; MacLean et al., Nature Rev. Microbiol., 7:287-296; U.S. Pat. Nos. 7,170,050; 7,302,146; 7,313,308; 7,476,503; all of which are herein incorporated by reference) utilizes reaction wells 50-100 nm in diameter and encompassing a reaction volume of approximately 20 zeptoliters (10⁻²¹ L). Sequencing reactions are performed using immobilized template, modified phi29 DNA polymerase, and high local concentrations of fluorescently labeled dNTPs. High local concentrations and continuous reaction conditions allow incorporation events to be captured in real time by fluor signal detection using laser excitation, an optical waveguide, and a CCD camera.

In certain embodiments, the single molecule real time (SMRT) DNA sequencing methods using zero-mode waveguides (ZMWs) developed by Pacific Biosciences, or similar methods, are employed. With this technology, DNA sequencing is performed on SMRT chips, each containing thousands of zero-mode waveguides (ZMWs). A ZMW is a hole, tens of nanometers in diameter, fabricated in a 100 nm metal film deposited on a silicon dioxide substrate. Each ZMW becomes a nanophotonic visualization chamber providing a detection volume of just 20 zeptoliters (10⁻²¹ L). At this volume, the activity of a single molecule can be detected amongst a background of thousands of labeled nucleotides. The ZMW provides a window for watching DNA polymerase as it performs sequencing by synthesis. Within each chamber, a single DNA polymerase molecule is attached to the bottom surface such that it permanently resides within the detection volume. Phospholinked nucleotides, each type labeled with a different colored fluorophore, are then introduced into the reaction solution at high concentrations which promote enzyme speed, accuracy, and processivity. Due to the small size of the ZMW, even at these high, biologically relevant concentrations, the detection volume is occupied by nucleotides only a small fraction of the time. In addition, visits to the detection volume are fast, lasting only a few microseconds, due to the very small distance that diffusion has to carry the nucleotides. The result is a very low background.

Processes and systems for such real time sequencing that may be adapted for use with the invention are described in, for example, U.S. Pat. No. 7,405,281, entitled “Fluorescent nucleotide analogs and uses therefor”, issued Jul. 29, 2008 to Xu et al.; U.S. Pat. No. 7,315,019, entitled “Arrays of optical confinements and uses thereof”, issued Jan. 1, 2008 to Turner et al.; U.S. Pat. No. 7,313,308, entitled “Optical analysis of molecules”, issued Dec. 25, 2007 to Turner et al.; U.S. Pat. No. 7,302,146, entitled “Apparatus and method for analysis of molecules”, issued Nov. 27, 2007 to Turner et al.; and U.S. Pat. No. 7,170,050, entitled “Apparatus and methods for optical analysis of molecules”, issued Jan. 30, 2007 to Turner et al.; and U.S. Pat. Pub. Nos. 20080212960, entitled “Methods and systems for simultaneous real-time monitoring of optical signals from multiple sources”, filed Oct. 26, 2007 by Lundquist et al.; 20080206764, entitled “Flowcell system for single molecule detection”, filed Oct. 26, 2007 by Williams et al.; 20080199932, entitled “Active surface coupled polymerases”, filed Oct. 26, 2007 by Hanzel et al.; 20080199874, entitled “CONTROLLABLE STRAND SCISSION OF MINI CIRCLE DNA”, filed Feb. 11, 2008 by Otto et al.; 20080176769, entitled “Articles having localized molecules disposed thereon and methods of producing same”, filed Oct. 26, 2007 by Rank et al.; 20080176316, entitled “Mitigation of photodamage in analytical reactions”, filed Oct. 31, 2007 by Eid et al.; 20080176241, entitled “Mitigation of photodamage in analytical reactions”, filed Oct. 31, 2007 by Eid et al.; 20080165346, entitled “Methods and systems for simultaneous real-time monitoring of optical signals from multiple sources”, filed Oct. 26, 2007 by Lundquist et al.; 20080160531, entitled “Uniform surfaces for hybrid material substrates and methods for making and using same”, filed Oct. 31, 2007 by Korlach; 20080157005, entitled “Methods and systems for simultaneous real-time monitoring of optical signals from multiple sources”, filed Oct. 26, 2007 by Lundquist et al.; 20080153100, entitled “Articles having localized molecules disposed thereon and methods of producing same”, filed Oct. 31, 2007 by Rank et al.; 20080153095, entitled “CHARGE SWITCH NUCLEOTIDES”, filed Oct. 26, 2007 by Williams et al.; 20080152281, entitled “Substrates, systems and methods for analyzing materials”, filed Oct. 31, 2007 by Lundquist et al.; 20080152280, entitled “Substrates, systems and methods for analyzing materials”, filed Oct. 31, 2007 by Lundquist et al.; 20080145278, entitled “Uniform surfaces for hybrid material substrates and methods for making and using same”, filed Oct. 31, 2007 by Korlach; 20080128627, entitled “SUBSTRATES, SYSTEMS AND METHODS FOR ANALYZING MATERIALS”, filed Aug. 31, 2007 by Lundquist et al.; 20080108082, entitled “Polymerase enzymes and reagents for enhanced nucleic acid sequencing”, filed Oct. 22, 2007 by Rank et al.; 20080095488, entitled “SUBSTRATES FOR PERFORMING ANALYTICAL REACTIONS”, filed Jun. 11, 2007 by Foquet et al.; 20080080059, entitled “MODULAR OPTICAL COMPONENTS AND SYSTEMS INCORPORATING SAME”, filed Sep. 27, 2007 by Dixon et al.; 20080050747, entitled “Articles having localized molecules disposed thereon and methods of producing and using same”, filed Aug. 14, 2007 by Korlach et al.; 20080032301, entitled “Articles having localized molecules disposed thereon and methods of producing same”, filed Mar. 29, 2007 by Rank et al.; 20080030628, entitled “Methods and systems for simultaneous real-time monitoring of optical signals from multiple sources”, filed Feb. 9, 2007 by Lundquist et al.; 20080009007, entitled “CONTROLLED INITIATION OF PRIMER EXTENSION”, filed Jun. 15, 2007 by Lyle et al.; 20070238679, entitled “Articles having localized molecules disposed thereon and methods of producing same”, filed Mar. 30, 2006 by Rank et al.; 20070231804, entitled “Methods, systems and compositions for monitoring enzyme activity and applications thereof”, filed Mar. 31, 2006 by Korlach et al.; 20070206187, entitled “Methods and systems for simultaneous real-time monitoring of optical signals from multiple sources”, filed Feb. 9, 2007 by Lundquist et al.; 20070196846, entitled “Polymerases for nucleotide analogue incorporation”, filed Dec. 21, 2006 by Hanzel et al.; 20070188750, entitled “Methods and systems for simultaneous real-time monitoring of optical signals from multiple sources”, filed Jul. 7, 2006 by Lundquist et al.; 20070161017, entitled “MITIGATION OF PHOTODAMAGE IN ANALYTICAL REACTIONS”, filed Dec. 1, 2006 by Eid et al.; 20070141598, entitled “Nucleotide Compositions and Uses Thereof”, filed Nov. 3, 2006 by Turner et al.; 20070134128, entitled “Uniform surfaces for hybrid material substrate and methods for making and using same”, filed Nov. 27, 2006 by Korlach; 20070128133, entitled “Mitigation of photodamage in analytical reactions”, filed Dec. 2, 2005 by Eid et al.; 20070077564, entitled “Reactive surfaces, substrates and methods of producing same”, filed Sep. 30, 2005 by Roitman et al.; 20070072196, entitled “Fluorescent nucleotide analogs and uses therefore”, filed Sep. 29, 2005 by Xu et al; and 20070036511, entitled “Methods and systems for monitoring multiple optical signals from a single source”, filed Aug. 11, 2005 by Lundquist et al.; and Korlach et al. (2008) “Selective aluminum passivation for targeted immobilization of single DNA polymerase molecules in zero-mode waveguide nanostructures” PNAS 105(4): 1176-81, all of which are herein incorporated by reference in their entireties.

The present disclosure contemplates any method capable of receiving, processing, and transmitting the information to and from laboratories conducting the assays, information provides, medical personal, and subjects. For example, in some embodiments of the present disclosure, a sample (e.g., a blood or EV sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., variant data), specific for the diagnostic or prognostic information desired for the subject.

The profile data is then prepared in a format suitable for interpretation by a treating clinician. For example, rather than providing raw expression data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action. In some embodiments, the results are used to select candidate therapies for drug screening or clinical trials.

Compositions for use in the methods described herein include, but are not limited to, kits comprising one or more reagents for determining the presence of mtDNA variants in a sample. In some embodiments, the reagents are, for example, one or more nucleic acid primers for the amplification, extension, or sequencing of the genes.

In preferred embodiments, the kits contain all of the components necessary to perform a detection assay, including all controls, directions for performing assays, and any necessary software for analysis and presentation of results.

In some preferred embodiments, the presence of specific variants described herein in a sample may be used to stratify subjects for and/or provide neo/adjuvant therapy (i.e., chemotherapy, radiotherapy, concurrent chemoradiotherapy (CRT), targeted therapy, immunotherapy, and combination thereof such as CRT plus chemotherapy). In some preferred embodiments, the neoadjuvant therapy is followed by surgery. Suitable neoadjuvant therapies include, but are not limited to, 5-Fluorouracil (5-FU), Capecitabine (Xeloda), Irinotecan (Camptosar), Oxaliplatin (Eloxatin), Trifluridine and Tipiracil (Lonsurf) as well as various radiotherapy regimens, immunotherapy regimens, or other biological agents as further described herein. Combination neoadjuvant therapies include, but are not limited to FOLFOX (fluorouracil, leucovorin, oxaliplatin), XELOX (capecitabine/oxalipatin), FOLFIRINOX (leucovorin [folinic acid], fluorouracil, irinotecan, and oxaliplatin) and CAPDX (capecitabine and oxaliplatin). In some embodiments, the neoadjuvant therapy comprises one or more chemotherapeutic agents (such as those just described) in combination with one or more immunotherapeutic agents. In some embodiments, the immunotherapeutic agent is a checkpoint inhibitor, such as a CTLA4, PD-1 or PD-L1 inhibitor. Suitable immunotherapeutic agents include, but not limited to, bevacizumab (inhibition of VEGF binding), cetuximab (EGFR inhibitor), panitumumab (EGFR inhibitor), ipilimumab (checkpoint inhibitor targeting CTLA4), nivolumab (checkpoint inhibitor targeting PD-1), pembrolizumab (checkpoint inhibitor targeting PD-1), toripalimab (checkpoint inhibitor targeting PD-1) and combinations thereof. Specific combination immuno/chemo neoadjuvant therapies include but are not limited to bevacizumab alone or in combination with 5-flurouracil/leucovorin/oxaliplatin and cetuximab/panitumumab alone or in combination with 5-flurouracil/leucovorin/oxaliplatin.

In some embodiments, neoadjuvant therapies may be followed by surgery and/or adjuvant therapy. Adjuvant therapies may be the same as those listed for neoadjuvant therapy and, most preferably, oxaliplatin, 5-fluorouracil, fluoropyrimidine, capecitabine, folinic acid, levimasole or a combination thereof, such as FOLFOX or CAPDX, alone or in further combination with an immunotherapeutic agent such as those listed above.

### Experimental

The following examples are provided in order to demonstrate and further illustrate certain preferred embodiments and aspects of the present invention and are not to be construed as limiting the scope thereof

### Example 1

### Methods

### Patients

Eligible patients have histologically confirmed CRC adenocarcinoma that is localized within the abdominal or pelvic cavity, which implies that the patients may be cured by surgery. In the current routine practice, assessment of metastatic risk is based on imaging modalities (e.g., CT and/or MR scanning).

### Blood Sampling

WB is collected by venipuncture on a tube containing an anticoagulant (e.g., EDTA, citrate) or the PAXgene Blood RNA Tube (Qiagen) at time of diagnosis. PaxGene blood is prepared according to the protocol of the vendor. Plasma is prepared from WB according to routine procedures (centrifugation at 1,000 g for 10 minutes). The specimens can be frozen and stored in accordance with routine procedures.

### Isolation of Plasma EVs

EVs are isolated from 100 μl plasma using qEV Size Exclusion chromatography Columns (IZON Science). The columns are equilibrated with 10 ml of 0.22-μm-filtered PBS and EVs are isolated according to the protocol of the vendor. 250-μ1 fractions are collected and the eluted fractions number 6-7 are treated with DNase (Sigma-Aldrich) and proteinase (Qiagen) prior to DNA isolation. When expedient, the isolated EVs are characterized according to our published procedures.¹⁴

Digital PCR Analysis of Selected mtDNA Sequences

This assay relies on the ability of a modification on the template DNA to inhibit restriction enzyme cleavage.^{17,18} A sequence containing the EarI and AgsI restriction enzyme sites in the MT-RNR2 gene is amplified using adapted forward and reverse primers (e.g., 5′-GATGGTGCAGCCGCTATTA-3′ (SEQ ID NO:1) and 5′-GGTGGGTGTGGGTATAATACTAAG-3′ (SEQ ID NO:2)) in the absence and presence of the enzymes. Samples can be partitioned using the QX200 Droplet Generator (Bio-Rad Laboratories) and analyzed with the QX200 Droplet Reader (Bio-Rad Laboratories). Data are given as the percentage of non-digested mtDNA [(mtDNA^{digested} copies per μl−mtDNA^{non-digested} copies per μl)×100].

The EarI restriction enzyme recognizes the wild-type (non-mutated) 3105 site of MT-RNR2, while the AgsI restriction enzyme recognizes a mutation in the 3106 site of MT-RNR2. Hence, a high percentage of non-digested product is expected by a PCR containing EarI if the mutation (point deletion) 3105AC>C is present, while for AgsI, the mutation (point deletion) 3106CN>C will cause a lower percentage of the non-digested product.

### Results

### Homoplasmic WB-mtDNA Variants

41 of the rectal cancer patients had been monitored for the absence or occurrence of metastatic events over 24-60 months of follow-up after completion of the curative-intent multimodal treatment. Their WB-mtDNA sequence data revealed that for each patient, two specific sites of the MT-RNR2 gene were homoplasmic for either the wild-type or a deletion (3105AC>A, 3106CN>C) variant, categorizing the patients into two groups that comprised the same cases for both mtDNA sites. The 41 patients comprised all 10 cases of metastatic progression, and all of the metastatic events occurred during the 18 first months of follow-up.

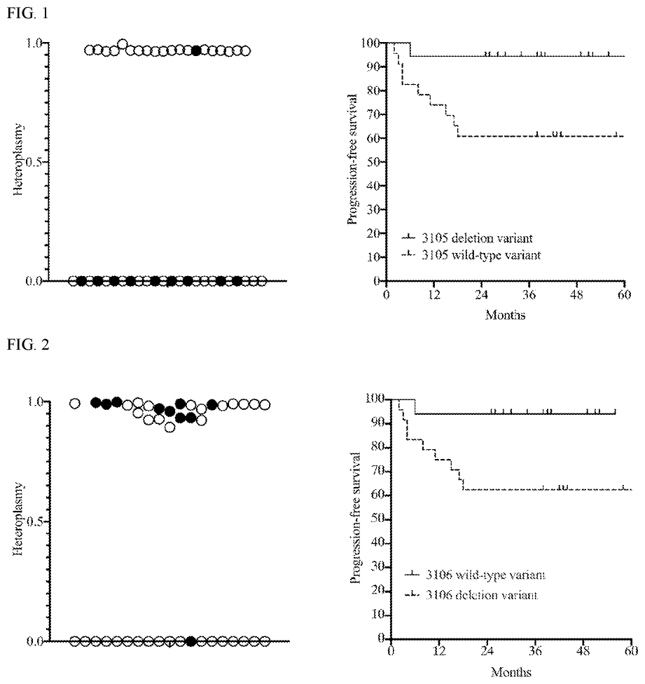

For the WB-mtDNA 3105 site, 23 patients had the wild-type homoplasmic variant and 18 patients were essentially homoplasmic (heteroplasmy of 0.964-0.995) for the AC>A deletion variant. Of the 10 metastatic events, 9 belonged to patients with the wild-type 3105 variant, while the single event in the deletion variant group appeared in a patient who had declined primary tumor surgery (i.e., refused the full curative-intent therapy) (FIG. 1). This gives an assay sensitivity of 56% and specificity of 90%

For the WB-mtDNA 3106 site, 18 patients had the wild-type homoplasmic variant and 23 patients were highly heteroplasmic (heteroplasmy of 0.893-0.998) for the CN>C deletion variant. Of the 10 metastatic events, 9 belonged to patients with the 3106 deletion variant, while the single event in the wild-type variant group appeared in the patient who had declined primary tumor surgery (FIG. 2). Assay sensitivity was 56% and specificity was 90%.

### REFERENCES

1. Dienstmann R et al. Nat Rev Cancer 2017; 17:79-92.
2. Punt C J et al. Nat Rev Clin Oncol 2017; 14:235-46.
3. Hadden W J et al. HPB (Oxford) 2016; 18:209-20.
4. Jung J et al. Nat Cell Biol 2019; 21:85-93.
5. Stewart J B, Chinnery P. Nat Rev Genet 2015; 16:530-42.
6. Sansone P et al. Proc Natl Acad Sci USA 2017; 114:E9066-75.
7. Torrabla D et al. Nat Commun 2018; 9:2658.
8. Aklilu M, Eng C. Nat Rev Clin Oncol 2011; 8:649-59.
9. Gerard J P et al. J Clin Oncol 2012, 30:4558-65.
10. Bosset J F et al. Lancet Oncol 2014; 15:184-90.
11. Rodel C et al. Lancet Oncol 2015; 16:979-89.
12. Breugom A J et al. Lancet Oncol 2015; 16:200-7.
13. Cercek A et al. JAMA Oncol 2018; 14:e180071.
14. Bjornetro T et al. J Extracell Vesicles 2019; 8:1567219.
15. Li H, Durbin R. Bioinformatics 2010; 26:589-95.
16. McKenna A et al. Genome Res 2010; 20:1297-303.
17. Wang W et al. Methods Mol Biol 2016; 1351:27-32.
18. Bousquet P A et al. Transl Oncol 2019; 12:76-83.

1. A method of identifying the presence of a mtDNA variant in a sample from a subject diagnosed with colorectal cancer (CRC), comprising:
a) contacting said sample with one or more reagents specific for detecting the presence of one or more variations in the MT-RNR2 gene; and
b) determining the presence of said variations in said sample.

2. The method of claim 1, wherein said variations are selected from the group consisting of 3105AC>A and 3106CN>C.

3. The method of claim 1, wherein said method comprises one or more of amplifying and/or sequencing said MT-RNR2 gene.

4. The method of claim 3, wherein said amplifying comprises digital PCR.

5. The method of claim 1, wherein said one or more reagents are selected from the group consisting of one or more sequencing primers, one or more amplification primers and one or more nucleic acid probes.

6. The method of claim 5, wherein said reagents further comprise one or more restriction enzymes.

7. The method of claim 1, wherein said sample is selected from the group consisting of whole blood (WB) and an isolated fraction of extracellular vesicles (EV).

8. The method of claim 1, wherein said subject has metastatic CRC.

9. A method of treating CRC, comprising:
a) determining the presence of one or more variations in the MT-RNR2 gene in a sample from a subject diagnosed with CRC, wherein said variations are selected from the group consisting of 3105AC>A and 3106CN>C; and
b) administering neo/adjuvant therapy to subjects with the absence of said 3105AC>A variation and/or the presence of said 3106CN>C variation.

10. The method of claim 9, wherein said determining comprises contacting said sample with one or more reagents specific for detecting the present of said variations.

11. The method of claim 9, wherein said method comprises one or more of amplifying and/or sequencing said MT-RNR2 gene.

12. The method of claim 11, wherein said amplifying comprises digital PCR.

13. The method of claim 9, wherein said one or more reagents are selected from the group consisting of one or more sequencing primers, one or more amplification primers and one or more nucleic acid probes.

14. The method of claim 13, wherein said reagents further comprise one or more restriction enzymes.

15. The method of claim 9, wherein said sample is selected from the group consisting of whole blood (WB) and an isolated fraction of extracellular vesicles (EV).

16. The method of claim 9, wherein said subject has metastatic CRC.

17. The method of claim 9, wherein said neo/adjuvant chemotherapy is selected from the group consisting of chemotherapy, radiotherapy, targeted therapy, and immunotherapy.

18. A method of determining an increased risk of a CRC patient having metastasis, comprising:
a) determining the presence of one or more variations in the MT-RNR2 gene in a sample from a subject diagnosed with CRC, wherein said variations are selected from the group consisting of 3105AC>A and 3106CN>C; and
b) identifying said subject as having an increased risk of metastasis when said sample has the absence of said 3105AC>A variation and/or the presence of said 3106CN>C variation.

19. The method of claim 18, wherein said determining comprises contacting said sample with one or more reagents specific for detecting the present of said variations.

20. The method of claim 18, wherein said method comprises one or more of amplifying and/or sequencing said MT-RNR2 gene.

21. The method of claim 20, wherein said amplifying comprises digital PCR.

22. The method of claim 18, wherein said one or more reagents are selected from the group consisting of one or more sequencing primers, one or more amplification primers and one or more nucleic acid probes.

23. The method of claim 22, wherein said reagents further comprise one or more restriction enzymes.

24. The method of claim 18, wherein said sample is selected from the group consisting of whole blood (WB) and an isolated fraction of extracellular vesicles (EV).

25. The method of claim 18, further comprising the step of administering adjuvant chemotherapy to said subjects with the absence of said 3105AC>A variation and/or the presence of said 3106CN>C variation.

26. The method of claim 25, wherein said neo/adjuvant chemotherapy is selected from the group consisting of chemotherapy, radiotherapy, targeted therapy, and immunotherapy.
